(19) United States
(12) Patent Application Publication
(10) Pub. No.: US 2013/0291860 A1
(43) Pub. Date: Nov. 7, 2013

(54) Method For Treating Cystic Fibrosis with Inhaled Denufosol

(71) Applicants: Ramesh Krishnamoorthy, Cary, NC (US); Carole Evans, Cary, NC (US); Brian J. Burke, Cary, NC (US); Amy E. Schaberg, Cary, NC (US); Thomas G. Coady, Apex, NC (US)

(72) Inventors: Ramesh Krishnamoorthy, Cary, NC (US); Carole Evans, Cary, NC (US); Brian J. Burke, Cary, NC (US); Amy E. Schaberg, Cary, NC (US); Thomas G. Coady, Apex, NC (US)

(21) Appl. No.: 13/878,929

(22) PCT Filed: Oct. 6, 2011
(86) PCT No.: PCT/US11/55121

§ 371 Date: Jul. 22, 2013

Related U.S. Application Data
(60) Provisional application No. 61/392,363, filed on Oct. 12, 2010.

(51) Int. Cl. A61K 9/00; A61M 16/14; A61K 31/7072

(52) U.S. Cl. CPC A61K 9/0078; A61K 31/7072; A61M 16/14
USPC 128/200.14

(57) ABSTRACT

The present invention is directed to a method for treating cystic fibrosis. The method comprises the steps of: identifying a patient suffering from cystic fibrosis, applying about 0.8-3 mL of a solution comprising about 18-65 mg/mL of denufosol into a medication reservoir of an nebulizer to achieve a target loading dose of denufosol of about 25-52 mg denufosol per dosing regimen, nebulizing the solution by passing through holes of an vibrating mesh device equipped with an oscillating membrane in the nebulizer, and delivering an inhaled respiratory dose of 20-33 mg to the lungs of the patient by inhalation within 3-9 minutes.

### TECHNICAL FIELD

This invention relates to methods of treating cystic fibrosis by administering a high concentration and a low volume of denufosol with an improved nebulizer to the lungs of a patient in a short period of time.

### BACKGROUND OF THE INVENTION

Cystic fibrosis (CF) is an autosomal recessive genetic disease, characterized by pulmonary and sinus disease, and gastrointestinal and reproductive tract dysfunction. The disease is caused by mutations in the cystic fibrosis transmembrane regulator (CFTR) gene, which encodes for an apical membrane epithelial protein that functions as a c-AMP-regulated chloride channel and a regulator of other channels. Defective CFTR results in abnormal ion transport and depleted airway surface liquid volume with reduced mucociliary clearance and a propensity for chronic infection of the respiratory tract with resulting inflammation, progressive airway damage and bronchiectasis. CF patients suffer from chronic repeated cycles of pulmonary bacterial colonization, pulmonary exacerbations and chronic lung function decline, which often lead to premature death. Although improved treatment of lung disease has increased survival, the median predicted age for survival is only 35 years, and patients continue to have significant morbidity, including hospitalizations.

Nucleotide P2Y₂ agonists, such as uridine 5-triphosphate (UTP) and diquafosol tetrasodium [P¹, P⁴-di(uridine 5′-) tetraphosphate, tetrasodium salt], regulate certain activities of the human airway epithelium. The P2Y₂ receptor is abundant on the luminal surface of polarized epithelial cells, especially those lining muscosal surfaces exposed to the external environment. P2Y₂ agonists act by stimulating the P2Y₂ receptor, which results in the secretion of chloride ion (C1′) and liquid and the inhibition of sodium (Na⁺) absorption to hydrate the airway surface liquid layer and to create a more normal periciliary fluid milieu. P2Y₂ agonists also act by stimulating mucin secretion from goblet cells and increasing ciliary beat frequency.

P2Y₂ receptor agonists represent a new approach to the treatment of CF, which bypasses the defective CFTR chloride channel, and activates an alternative chloride channel. This activation results in an increase in airway surface epithelial hydration, and through these actions and effects on cilia beat frequency, increases mucociliary clearance. Denufosol tetrasodium [P¹- (uridine 5″-)-P⁴-(2′-deoxycytidine 5′-) tetraphosphate, tetrasodium salt], a chemically stable, selective P2Y₂ receptor agonist, has been investigated in clinical trial studies as a treatment for patients with CF. (Kellerman, et al., Pulm Pharmacol Ther., 21: 600-7, 2008; Deterding, et al., Pediatric Pulm 39: 339-348, 2005; Yerxa, et al., J. Pharmacol Exo Ther., 302: 871-880, 2002).

There is a need for an improved method for treating cystic fibrosis; such method is not only effective to treat cystic fibrosis but also reduces the treatment time and improves patient compliance.

### SUMMARY OF THE INVENTION

The present invention is directed to a method for treating cystic fibrosis with inhaled denufosol. The method comprises the steps of: identifying a patient suffering from cystic fibrosis, applying about 0.8-3 mL of a solution comprising about 18-65 mg/mL of denufosol into a medication reservoir of an nebulizer to achieve a target loading dose of denufosol of about 25-52 mg denufosol per dosing regimen, nebulizing the solution by passing through holes of an vibrating mesh device equipped with an oscillating membrane in the nebulizer, generating the aerosol particles from the nebulizer at an output rate of 0.25-0.5 mL/minute, and delivering an inhaled respiratory dose of 20-33 mg of denufosol to the lungs of the patient by inhalation within 3-9 minutes. Preferred denufosol is denufosol tetrasodium.

### DETAILED DESCRIPTION OF THE INVENTION

The inventors have discovered an effective method for treating cystic fibrosis (CF) by administering denufosol in an aerosol form to the lung of a patient suffering from CF. The present method significantly reduces the administration time for denufosol and reduces the unnecessary waste of the drug, while at the same time improves the overall efficiency of the aerosolization process. The present invention improves the quality of life and patient compliance. The present invention also provides a significant economic benefit due to reduced loss of the drug in the medication reservoir and provides an improvement in treatment time.

The present invention is directed to a method for treating cystic fibrosis. The method comprises the steps of: identifying a human patient suffering from cystic fibrosis, applying about 0.8-3 mL of a solution comprising about 18-65 mg/mL of denufosol into a medication reservoir of an nebulizer to achieve a target loading dose of denufosol of about 25-52 mg denufosol per dosing regimen, nebulizing the solution by passing through holes of an vibrating mesh device equipped with an oscillating membrane in the nebulizer, generating the aerosol particles from the nebulizer at an output rate of 0.25-0.5 mL/minute, and delivering an inhaled respiratory dose of 20-33 mg of denufosol to the lungs of the patient by inhalation within 3-9 minutes. The aerosol particles generated preferably have a mass median aerodynamic diameter between about 2.5-4.5 μm with a geometric standard deviation of 1.2-1.8, which can effectively reach the lungs of a CF patient.

The above method is applied to a patient once a day or twice a day or three times a day, such that an effective amount of denufosol is delivered to the patient daily. “An effective amount” as used herein, is meant an amount that has a therapeutic effect, which improves the lung function, as measured by FEV1 of the patient being treated “About” as used in this application, refers to ±10% of the recited value.

Denufosol

The chemical name of denufosol is P¹-(uridine 5′-)-P⁴-(2′-deoxycytidine tetraphosphate; its chemical registry number is 211448-85-0. Denufosol is a P2Y₂ receptor agonist, which has the ability to restore or maintain mucociliary clearance in patients relatively early in the CF lung disease process, thus preserving lung function and lessening the inevitable repeat cycles of pulmonary bacterial colonization, pulmonary exacerbations, and chronic lung function decline.

Denufosol of the present invention encompasses its pharmaceutically acceptable salts, such as, but not limited to, an alkali metal salt such as sodium or potassium; an alkaline earth metal salt such as manganese, magnesium or calcium; or an ammonium or tetraalkyl ammonium salt. Pharmaceutically acceptable salts are salts that retain the desired biological activity of the parent compound and do not impart undesired toxicological effects.

Pharmaceutically acceptable salts of denufosol include tetra-(alkali metal) salts, wherein the alkali metal is sodium, potassium, lithium, or the combination thereof. For example, tetra-(alkali metal) salts of denufosol include tetrasodium salts, tetrapotassium salts, tetralithium salts, trisodium/monopotassium salts, disodium/dipotassium salts, monosodium/tripotassium salts, trisodium/monolithium salts, disodium/dilithium salts, monosodium/trilithium salts, disodium/monopotassium/monolithium salts, dipotassium/monosodium/monolithium salts, and dilithium/monosodium/monopotassium salts. Tetrasodium salt is a preferred salt.

Other pharmaceutically acceptable salts of denufosol include tetraammonium salts and tetra(quaternary ammonium) salts.

Routes of Administration

The key of the invention lies in the ability to efficiently deliver denufosol in a high concentration and in a small volume to the lungs. Any local administration method for delivering denufosol to the lumen of the lung is suitable for the present invention.

Local administration includes inhalation, topical application, and targeted drug delivery. Methods of inhalation include liquid instillation, inhalation of aerosolized solution or pressurized fluid preparation via nebulizer (most preferred), inhalation of dry powder or a mixture of ingredients in a fluid formulation by inhaler (more preferred), and directing soluble or dried material of soluble or insoluble fractions of a discrete particle size distribution into the air stream during mechanical ventilation (preferred).

An example of targeted drug delivery is enclosure of denufosol within a liposome, where the liposome is coated with a specific antibody whose antigen is expressed in the targeted lung tissue.

Another example of a delivery system includes nanoparticulate or microparticulate compositions of denufosol. In such a case, denufosol is formulated as a nanosuspension with the carrier loaded with the compounds; such a preparation is then filtered through a fine porous membrane or a suitable filtering medium, or is exposed to solvent interchanges to produce nanoparticles. Such nanoparticulate preparations are freeze-dried or held in suspension in an aqueous or physiologically compatible medium. The preparations so obtained can be inhaled by suitable means.

Another example of a suitable preparation includes a reconstitutable preparation. In this case, denufosol is formulated in a preparation to contain the necessary adjuvants to make it physiologically compatible. Such a preparation is reconstituted by addition of water or suitable physiological fluids, admixed by simple agitation and inhaled using appropriate techniques.

Denufosol can be prepared into dry powder or equivalent inhalation powders using the well known art of super critical fluid technology. In such a case, denufosol is admixed with appropriate excipients and milled into a homogenous mass using suitable solvents or adjuvants. Following this, this mass is subjected to mixing using super critical fluid technology to achieve suitable particle size distribution. The desired particle size is the size suitable for direct inhalation into the lungs using a suitable inhalation technique, or the size suitable for being introduced into the lungs via a mechanical ventilator. Alternatively, the size is large enough to be admixed with a fluid, wherein the particle dissolves mostly or completely prior to nebulization into the lungs.

To prevent particle size growth and minimize crystal growth, the particle can be spray-dried to have better aerodynamic properties than micronized material.

Another example of a suitable preparation includes a preparation of freeze-dried or lyophilized preparation of denufosol. Such a preparation is made to protect the inherent instability of the molecule due to physical or chemical changes induced in the presence of certain solvents or processing techniques. Cryoprotectants can be used to further maintain the physical and chemical stability of denufosol. The lypophilized preparations can be used as is in the form of a dry powder inhaler. The lypophilized preparations can also be admixed with other suitable adjuvants and be used as dry powder inhaler or as nebulized preparation.

In one embodiment, denufosol is administered 1, 2, or 3 times a day. In general, denufosol is administered at 20-100 mg per dosage, or 25-90 mg per dosage, or 30-60 mg per dosage, preferred 25-52 mg per dosage, when administered one to three times a day, preferably administered two times a day.

Pharmaceutical Formulation

The present invention administers to the patient a pharmaceutical formulation comprising denufosol or a pharmaceutically acceptable salt thereof and a pharmaceutically acceptable carrier. Preferred denufosol is denufosol tetrasodium.

The pharmaceutical formulation of the present invention is in a liquid form or in a form of an inhalable dry powder. A liquid form is preferred.

When in a liquid form, the pharmaceutical formulation comprises about 18-65, or 18-50, 20-45, or 22-35 mg/mL of denufosol tetrasodium. In one embodiment, the pharmaceutical formulation comprises about 20-60 mg/mL, preferably 20-45 mg/mL of denufosol tetrasodium in about 0.8-3 mL, which can be administered to CF patient as a single dosage unit in the form of aerosol for oral inhalation. For example, the pharmaceutical formulation comprises about 20-45 mg of denufosol tetrasodium in about 1-3 mL, preferably about 1.5-2.8 mL.

Pharmaceutically acceptable carriers include excipients, diluents, salts, buffers, stabilizers, solvents, isotonic agents, and other materials known in the art. The pharmaceutical formulation optionally includes potentiators, targeting agents, stabilizing agents, cosolvents, pressurized gases, or solubilizing conjugates.

Acceptable excipients include sugars such as lactose, sucrose, mannitol, or sorbitol; cellulose preparations such as, maize starch, wheat starch, rice starch, potato starch, gelatin, gum tragacanth, methyl cellulose, hydroxypropylmethylcellulose, sodium caroxymethylcellulose, and/or polyvinylpyrrolidone (PVP). Preferred excipients include lactose, gelatin, sodium carboxymethyl cellulose, and low molecular weight starch products.

Acceptable suspending agents that can serve as valve lubricants in pressurized pack inhaler systems are desirable. Such agents include oleic acid, simple carboxylic acid derivatives, and sorbitan trioleate.

Acceptable diluents include water, saline, phosphate-buffered citrate or saline solution, and mucolytic preparations. Other diluents that can be considered include alcohol, propylene glycol, and ethanol; these solvents or diluents are more common in oral aerosol formulations. Physiologically acceptable diluents that have a tonicity and pH compatible with the alveolar apparatus are desirable. Preferred diluents include isotonic saline, phosphate buffered isotonic solutions whose tonicity have been adjusted with sodium chloride or sucrose or dextrose or mannitol.

Acceptable fillers include glycerin, propylene glycol, and ethanol in liquid or fluid preparations. Suitable fillers for dry powder inhalation systems include lactose, sucrose, dextrose, suitable amino acids, and derivatives of lactose. Preferred fillers include glycerin, propylene glycol, lactose and certain amino acids.

Acceptable salts include those that are physiologically compatible and provide the desired tonicity adjustment. Monovalent and divalent salts of strong or weak acids are desirable. Preferred salts include sodium chloride, sodium citrate, ascorbates, and sodium phosphates.

Acceptable buffers include phosphate or citrate buffers or mixed buffer systems of low buffering capacity. Preferred buffers include phosphate or citrate buffers.

Acceptable stabilizers include those that provide chemical or physical stability of the final preparations. Such stabilizers include antioxidants such a sodium metabisulfite, alcohol, polyethylene glycols, butylated hydroxyanisole, butylated hydroxytoluene, disodium edetate. Preferred stabilizers include sodium metabisulfite, disodium edetate and polyethylene glycols. Included within this class of stabilizers would be cryoprotectants such as polyethylene glycols, sugars, and carrageenans.

Acceptable solubilizers include propylene glycol, glycerin, suitable amino acids, and complexing agents such as cyclodextrins, sorbitol solution, or alcohol. Solubilizers including ethanol, propylene glycol, glycerin, sorbitol, and cyclodetrins are desirable. Preferred solubilizers include propylene glycol, sorbitol, and cyclodextrins.

The active ingredients can be formulated for inhalation with use of a suitable propellant such as dichlorodifluoromethane, dichloroflouromethane, dichlorotetrafluoroethane, carbon dioxide or other gas. Preferred propellants include non-CFC related class of propellants or related analogs.

The active ingredients can also be dried into an inhalable dry powder. This can be achieved by mixing with suitable adjuvants that are compatible with denufosol and offer biological compatibility. Desirable methods of drying the pharmaceutical material for inhalation include spray drying, conventional bed drying, or super critical fluid processing; with spray drying and super critical fluid processing being preferred.

When in an inhalable dry powder form, the pharmaceutical formulation comprises about 30-90, or 40-80, or 50-70 mg of denufosol tetrasodium in a unit dosage form. For example, the pharmaceutical formulation comprises about 60 mg of denufosol tetrasodium in a unit dosage form.

Device for Delivery of Aerosolized Denufosol Solution

A nebulizer is selected primarily on the basis of allowing the formation of denufosol aerosol having a majority of mass median aerodynamic diameter (MMAD)) between 2.5 to 5 μm, preferably about 2.5-4.5 μm, preferably about 2.8-4 μm, or preferably about 3-4 gm. The amount of denufosol delivered to the lung must be efficacious for treating CF. If an aerosol contains a large number of particles with a MMAD larger than 5 μm, the particles are deposited in the upper airways decreasing the amount of denufosol delivered to the lung. If an aerosol contains a large number of particles with a MMAD less than 1 μm, the particles are not deposited in the peripheral lung but they continues to be delivered into the alveoli and may get transferred into the systemic blood circulation.

Nebulizer suitable for practicing this invention must be able to nebulize a small volume (0.8-3 mL) of the formulation efficiently into aerosol particles, at a size range predominantly from 2.5 to 4.5 μm, preferably 2.8 to 4 μm. Predominantly in this application means that at least 70% but preferably more than 90% of all generated aerosol particles are within 2.5 to 4.5 μm, preferably 2.8 to 4 μm.

Typical nebulizing devices suitable for practicing this invention include atomizing nebulizers, or modified jet nebulizers, ultrasonic nebulizers, electronic nebulizers, vibrating porous plate nebulizers, and energized dry powder inhalers modified for handling small volume of highly concentrated drug. An atomized nebulizer engages an aerosol generator to produce atomized aerosol. A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating mesh (porous plate) device utilizes rapid vibration to shear a stream of liquid into appropriate droplet sizes. A preferred device is a vibrating mesh nebulizer that is suitable for handling small volumes of aqueous solution preparations.

Typically, nebulizers that utilize the vibrating mesh technology are capable of delivering aerosol droplets that have a much narrower droplet size distribution compared to that of a conventional jet nebulizer. The narrow droplet size distribution allows a more efficient target delivery to the lungs of a patient, thus improving the overall efficiency of the drug delivery to the lungs and ultimately improving the patient's quality of life.

By using nebulizers that utilize vibrating mesh technology, a loading dose that is put into the medication reservoir can be reduced compared to that dose used in a jet nebulizer and yet deliver a comparable amount of drug to the lungs. This improvement is due to a greater efficiency of the vibrating mesh nebulizers for converting the drug solution into aerosol particles and delivering to a patient. The loading dose of denufosol in the present invention is ≦85%, preferably ≦80%, 70%, 60%, or 50% of that used in a current denufosol delivery system.

Nebulizers using the vibrating mesh technology include the following: a modified Aeroneb® Go, manufactured by Aerogen, with a modified device body and membrane; the eFlow® System, manufactured by PARI, with an oscillating membrane (see U.S. Pat. Nos. 7,458,372, 7,472,401, 6,962,151, and 7,252,085). These devices aerosolize liquid by extruding the liquid through an oscillating membrane containing hundreds of small holes. The droplet size of the emitted aerosol is controlled by the dimensions of the holes in the membrane. In order for the delivered aerosol to closely match the droplet size distribution ideally suited for denufosol formulation, the membrane in the candidate devices may need to be modified. Thus, the final selected device is a device that specifically matches the denufosol delivery parameters as described in this invention, i.e., the aerosol particles obtained having a mass median aerodynamic diameter between about 2.5-4.5 μm with a geometric standard deviation of 1.2-1.8, and the aerosol particles generated from the nebulizer are at an output rate of 0.25-0 5 mL/minute.

The nebulizer contains a liquid storage container (medication reservoir). For administration of the denufosol solution, about 0.8 to 3 ml, preferably 0.9-2.8 mL, 1-2.8 mL, or 1-2.5 mL, of the denufosol formulation is placed in the storage container, and then an aerosol of particle sizes between 3 and 4.5 μm are produced.

A high concentration of denufosol formulation and an effective nebulizing device significantly enhance the efficiency and speed of drug administration. Currently, the average time for administration of aerosolized denufosol is about 15 minutes per dosing regimen, and it is administered three times per day. The present method places a high concentration of denufosol in the medication reservoir, i.e., 18-65 mg/mL, or 20-45 mg/mL, or 22-35 mg/mL of denufosol. The present invention delivers aerosolized denufosol in about 2-9 minutes per dosing regimen, preferably about 3-8 minutes per dosing regimen, and most preferably about 4-7 minutes per dosing regimen, which significantly reduces the time required for the treatment and increases patient compliance.

The present invention utilizes an efficient nebulizer system, which reduced the amount of the denufosol solution remaining in the nebulizer at the end of treatment, thus reducing the waste of medication. For example, the amount of the denufosol solution remaining in the nebulizer at the end of treatment is ≦45%, or ≦30%, or ≦20%, or ≦10% of the amount of the starting solution. In other words, the efficiency of converting the denufosol into aerosol particles from the medication reservoir and delivering it to the patient is ≧55%, ≧70%, or ≧80%, or ≧90%. For example, when 2.7 mL of the denufosol solution is applied to the medication reservoir, only about 0.3 mL of the solution remains in the nebulizer at the end of treatment.

The effective nebulizer, with an output (aerosol particles generated) of about 0.25 to 0.6 mL/minute, preferably about 0.25 to 0.5 mL/minute, 0.3-0.5 mL/minute, or 0.3-0.45 mL/minute, is capable of quickly delivering a drug material. In a preferred embodiment, the nebulizer is able to aerosolize about 90% of the denufosol placed in the nebulization chamber, with 85% or more of the aerosol particles being within the size range required for lung deposition. As a result, administration of a high concentration of denufosol solution using an effective nebulizer leads to substantial improvement in local delivery to the lung, which reduces treatment time to as little as about 4-7 minutes.

CF patients in general have a low inspiratory flow rate of 15-20 L/minute, compared with normal people's approximately 25-30 L/minute. The present method of the delivery of the solution using the said device(s) delivers denufosol efficiently to the lungs of a patient and is not significantly impacted by the low inspiratory flow rate of the CF patient.

The invention is illustrated further by the following examples that are not to be construed as limiting the invention in scope to the specific procedures described in them.

### EXAMPLES

### Example 1

Test Design

Patients are enrolled and randomly assigned to receive denufosol tetrasodium or placebo one to three times a day. Patients are instructed to inhale study drug (denufosol tetrasodium) or placebo using a vibrating mesh nebulizer (e.g. PARI eFLOW® Nebulizer system or equivalent), loaded with about 50 mg of denufosol tetrasodium inhalation solution (see Table 1) or placebo. At the end of the 24-week double-blind, placebo-controlled treatment period, placebo patients receive about 50 mg denufosol tetrasodium as a loading dosage for a 24-week safety extension period. All patients on denufosol tetrasodium during the first 24 weeks continue to receive denufosol tetrasodium during the 24-week safety extension. Upon completion of study participation or discontinuation from study treatment, all patients are scheduled for a 1-week follow-up visit.

Subjects

Subjects are ≧5 years of age and had a confirmed diagnosis of CF (positive sweat chloride value >60 mEq/L, and/or genotype with two identifiable mutations consistent with CF, accompanied by one or more clinical features consistent with the CF phenotype). Subjects have a forced expiratory volume at one second (FEV1) >75% of predicted normal for age, gender, and height.

In general, CF patients take many medications as their usual standard of care. This study is designed to randomly assign patients either to denufosol or placebo on top of their usual standard of care. Patients are instructed whenever possible to use these medications consistently throughout the study.

Denufosol Formulations

Listed below in Table 1 are some prophetic examples of denufosol tetrasodium inhalation solutions of various strengths that may be prepared. These formulations of denufosol are all sterile, aqueous solutions that may be used in conjunction with the drug delivery device mentioned herein.

TABLE 1
Composition of denufosol tetrasodium Inhalation Solutions

| Ingredient | 20 mg/mL | 30 mg/mL | 45 mg/mL |
|---|---|---|---|
| Denufosol tetrasodium | 20 mg/mL | 30 mg/mL | 45 mg/mL |
| Sodium Chloride, USP/EP | 6.6 mg/mL | 5.4 mg/mL | 3.5 mg/mL |
| Water for Injection, USP/EP | q.s. ad 100% | q.s. ad 100% | q.s. ad 100% |
| Adjust pH to | 7.3 ± 0.2 | 7.3 ± 0.2 | 7.3 ± 0.2 |

Test Protocols

All patients are instructed to inhale the study drug using normal tidal breathing via the vibrating mesh nebulizer system (e.g. PARI eFLOW® Nebulizer system or equivalent). A dose is considered complete after about 6 minutes of inhalation. Study drug is suggested to be taken TID at the same time each day.

Assessment of Efficacy

Primary efficacy endpoint is change in lung function, as measured by FEV1 (L), from baseline to Week 24 end point. Secondary efficacy endpoints included the following: time to first pulmonary exacerbation during the 24-week placebo-controlled treatment period; incidence of pulmonary exacerbations during the 24-week placebo-controlled treatment period; number of pulmonary exacerbations/time at risk (incidence density) during the 24-week placebo-controlled treatment period; change in lung function, as measured by FEV1 (L) from baseline to Weeks 4 and 12, and FVC (L) and FEF 25%-75% (L/sec) from baseline to Weeks 4, 12, 24, and the end point. Other secondary efficacy endpoints include incidence of IV antibiotic use during the 24-week placebo-controlled treatment period; number of days of IV antibiotic use during the 24-week placebo-controlled treatment period; incidence of new use of antipseudomonal antibiotics during the 24-week placebo-controlled treatment period; incidence of hospitalizations/ER visits for a respiratory-related complaint during the 24-week placebo-controlled treatment period; number of days spent in the hospital for a respiratory-related complaint during the 24-week placebo-controlled treatment period; changes from baseline to Weeks 12 and 24 in Health-related Quality of Life as measured by the Cystic Fibrosis Questionnaire and the Feeling Thermometer; and changes in utility assessment from baseline to Weeks 12 and 24 as measured by the Health Utilities Index; number of CF-related days lost from work or school during the 24-week placebo-controlled treatment period; and responses at Week 24 to the Patient Questionnaire.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications can be made therein without departing from the scope of the present invention as set forth in the claims. To particularly point out and distinctly claim the subject matter regarded as invention, the following claims conclude this specification.

What is claimed is:

1. A method for treating cystic fibrosis, comprising the steps of:
identifying a patient suffering from cystic fibrosis,
applying 0.8-3 mL of a solution comprising 18-65 mg/mL of denufosol into a medication reservoir of a nebulizer to achieve a target loading dose of denufosol of about 25-52 mg denufosol per dosing regimen,
nebulizing the solution by passing through holes of a vibrating mesh device equipped with an oscillating membrane in the nebulizer,
generating the aerosol particles from the nebulizer at an output rate of 0.25-0.5 mL/minute, and
delivering an inhaled respiratory dose of 20-33 mg to the lungs of the patient by inhalation within 3-9 minutes.

2. The method according to claim 1, wherein said denufosol is denufosol tetrasodium.

3. The method according to claim 1, wherein said solution comprises 20-45 mg/mL of denufosol tetrasodium.

4. The method according to claim 1, wherein 0.9-2.8 mL of the solution is applied into the medication reservoir.

5. The method according to claim 1, wherein said denufosol is delivered at an inhaled respiratory dose of about 20-30 mg to the lungs of the patient in 4-7 minutes.

6. The method according to claim 1, wherein the aerosol particles generated have a mass median aerodynamic diameter of about 2.5-4.5 μm with a geometric standard deviation of about 1.2-1.8.

7. The method according to claim 1, wherein the aerosol particles generated have a mass median aerodynamic diameter of about 3-4 μm.
